(19) United States
(12) Patent Application Publication
(10) Pub. No.: US 2007/0255098 A1
(43) Pub. Date: Nov. 1, 2007

(54) System and method for in vivo imager with stabilizer

(75) Inventors: Kang-Huai Wang, Saratoga, CA (US); Gordon Wilson, Saratoga, CA (US)

Correspondence Address: STEVENS LAW GROUP, P.O. BOX 1667, SAN JOSE, CA, 95109, US

(73) Assignee: Capso Vision, Inc., Saratoga, CA (US)

(21) Appl. No.: 11/655,640

(22) Filed: Jan. 19, 2007

Related U.S. Application Data
(60) Provisional application No. 60/760,794, filed on Jan. 19, 2006.

(51) Int. Cl. A61B 1/00

(52) U.S. Cl. 600/109

(57) ABSTRACT

A swallowable capsule with a camera and a memory for imaging the colon. Standard semiconductor memory (memories made of standard memories processes or processes modified from standard process by adopting comprehensible silicon planar technology process steps) is used. This is made possible by the use of an optimal type of image compression that can be performed with limited processing power and limited memory (e.g., without requiring a full size frame buffer). Also, controls on the number of images taken are used in one embodiment.

### BACKGROUND OF THE INVENTION

Various autonomous devices have been developed that are configured to capture an image from within in vivo passages and cavities within a body, such as those passages and cavities within the gastrointestinal (GI) tract. These devices typically comprise a digital camera housed within a capsule along with light sources for illumination. The capsule may be powered by batteries or by inductive power transfer from outside the body. The capsule may also contain memory for storing captured images and/or a radio transmitter for transmitting data to an ex vivo receiver outside the body.

A common diagnostic procedure involves the patient swallowing the capsule, whereupon the camera begins capturing images and continues to do so at intervals as the capsule moves passively through the cavities made up of the inside tissue walls of the GI tract under the action of peristalsis. The capsule's value as a diagnostic tool depends on it capturing images of the entire interior surface of the organ or organs of interest. Unlike endoscopes, which are mechanically manipulated by a physician, the orientation and movement of the capsule camera are not under an operator's control and are solely determined by the physical characteristics of the capsule, such as its size, shape, weight, and surface roughness, and the physical characteristics and actions of the bodily cavity. Both the physical characteristics of the capsule and the design and operation of the imaging system within it must be optimized to minimize the risk that some regions of the target lumen are not imaged as the capsule passes through the cavity.

Two general image-capture scenarios may be envisioned, depending on the size of the organ imaged. In relatively constricted passages, such as the esophagus and the small intestine, a capsule which is oblong and of length less than the diameter of the passage, will naturally align itself longitudinally within the passage. Typically, the camera is situated under a transparent dome at one (or both) ends of the capsule. The camera faces down the passage so that the center of the image comprises a dark hole. The field of interest is the intestinal wall at the periphery of the image

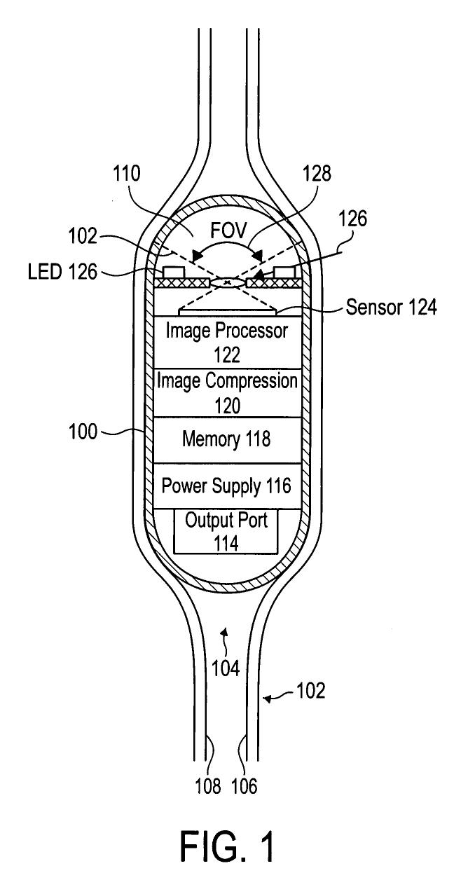

FIG. 1 illustrates a capsule camera in the prior art. The capsule 100 is encased in a housing 101 so that it can travel in vivo inside an organ 102, such as an esophagus or a small intestine, within an interior cavity 104. The capsule may be in contact with the inner surfaces 106,108 of the organ, and the camera lens opening 110 can capture images within its field of view 112. The capsule may include an output port 114 for outputting image data, a power supply 116 for powering components of the camera, a memory 118 for storing images, image compression 120 circuitry for compressing images to be stored in memory, an image processor 122 for processing image data, and LEDs 126 for illuminating the surfaces 106,108 so that images can be captured from the light that is scattered off of the surfaces.

It is desirable for each image to have proportionally more of its area to be intestinal wall and proportionally less the receding hole in the middle. Thus, a large FOV is desirable. A typical FOV is 140°. Unfortunately, a simple wide-angle lens will exhibit increased distortion and reduced resolution and numerical aperture at large field angles. High-performance wide-angle and “fish-eye” lenses are typically large relative to the aperture and focal length and consist of many lens elements. A capsule camera is constrained to be compact and low-cost, and these types of configurations are not cost effective. Further, these conventional devices waste illumination at the frontal area of these lenses, and thus the power used to provide such illumination is also wasted. Since power consumption is always a concern, such wasted illumination is a problem. Still further, since the intestinal wall within the filed of view extends away from the capsule, it is both foreshortened and also requires considerable depth of field to image clearly in its entirety. Depth of field comes at the expense of exposure sensitivity.

The second scenario occurs when the capsule is in a cavity, such as the colon, whose diameter is larger than any dimension of the capsule. In this scenario the capsule orientation is much less predictable, unless some mechanism stabilizes it. Assuming that the organ is empty of food, feces, and fluids, the primary forces acting on the capsule are gravity, surface tension, friction, and the force of the cavity wall pressing against the capsule. The cavity applies pressure to the capsule, both as a passive reaction to other forces such as gravity pushing the capsule against it and as the periodic active pressure of peristalsis. These forces determine the dynamics of the capsule's movement and its orientation during periods of stasis. The magnitude and direction of each of these forces is influenced by the physical characteristics of the capsule and the cavity. For example, the greater the mass of the capsule, the greater the force of gravity will be, and the smoother the capsule, the less the force of friction. Undulations in the wall of the colon will tend to tip the capsule such that the longitudinal axis of the capsule is not parallel to the longitudinal axis of the colon.

Also, whether in a large or small cavity, it is well known that there are sacculations that are difficult to see from a capsule that only sees in a forward looking orientation. For example, ridges exist on the walls of the small and large intestine and also other organs. These ridges extend somewhat perpendicular to the walls of the organ and are difficult to see behind. A side or reverse angle is required in order to view the tissue surface properly. Conventional devices are not able to see such surfaces, since their FOV is substantially forward looking. It is important for a physician to see all areas of these organs, as polyps or other irregularities need to be thoroughly observed for an accurate diagnosis. Since conventional capsules are unable to see the hidden areas around the ridges, irregularities may be missed, and critical diagnoses of serious medical conditions may be flawed. Thus, there exists a need for more accurate viewing of these often missed areas with a capsule.

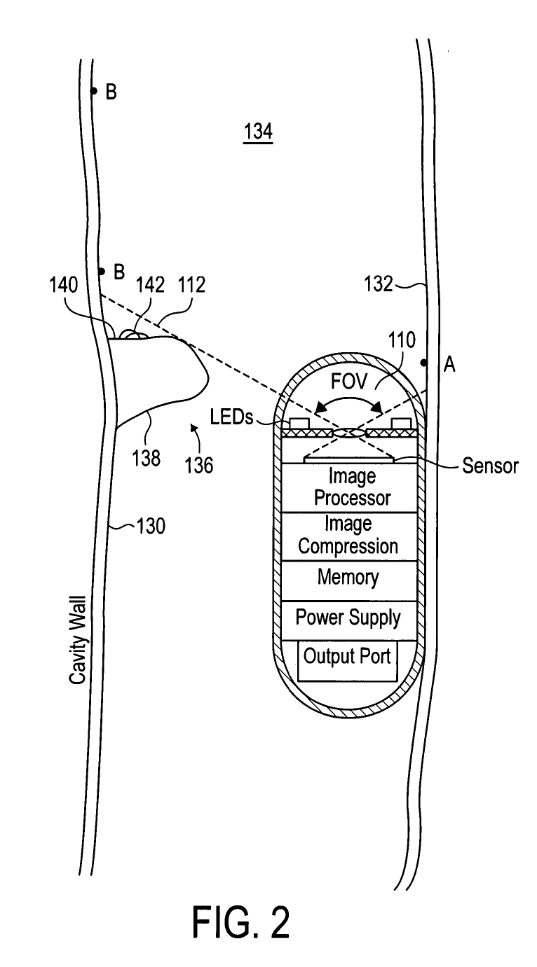

FIG. 2 shows a relatively straightforward example where the passage 134, such as a human colon, is relatively horizontal, with the exception of the ridge 136, and the capsule sits on its bottom surface 132 with the optical axis of the camera parallel to the colon longitudinal axis. The ridge illustrates a problematic viewing area as discussed above, where the front surface 138 is visible and observable by the capsule 100 as it approaches the ridge. The backside of the capsule 140, however, is not visible by the capsule lens, as the limited FOV 110 does not pick up that surface. Specifically, the range 110 of the FOV misses part of the surface, and moreover misses the irregularity illustrated as polyp 142.

Three object points within the field of view 110 are labeled A, B, and C. The object distance is quite different for these three points, where the range of the view 112 is broader on one side of the capsule than the other, so that a large depth of field is required to produce adequate focus for all three simultaneously. Also, if the LED (light emitting diode) illuminators provide uniform flux across the angular FOV, then point A will be more brightly illuminated than point B and point B more than point C. Thus, an optimal exposure for point B results in over exposure at point A and under exposure at point C. For each image, only a relatively small percentage of the FOV will have proper focus and exposure, making the system inefficient. Power is expended on every portion of the image by the flash and by the imager, which might be an array of CMOS or CCD pixels. Moreover, without image compression, further system resources will be expended to store or transmit portions of images with low information content. In order to maximize the likelihood that all surfaces within the colon are adequately imaged, a significant redundancy, that is, multiple overlapping images, is required.

One approach to alleviating these problems is to reduce the instantaneous FOV but make the FOV changeable. Patent application 2005/0146644 discloses an in-vivo sensor with a rotating field of view. The illumination source may also rotate with the field of view so that regions outside the instantaneous FOV are not wastefully illuminated. This does not completely obviate the problem of wasteful illumination, and furthermore creates other power demands when rotating. Also, this innovation by itself does not solve the depth of field and exposure control problems discussed above.

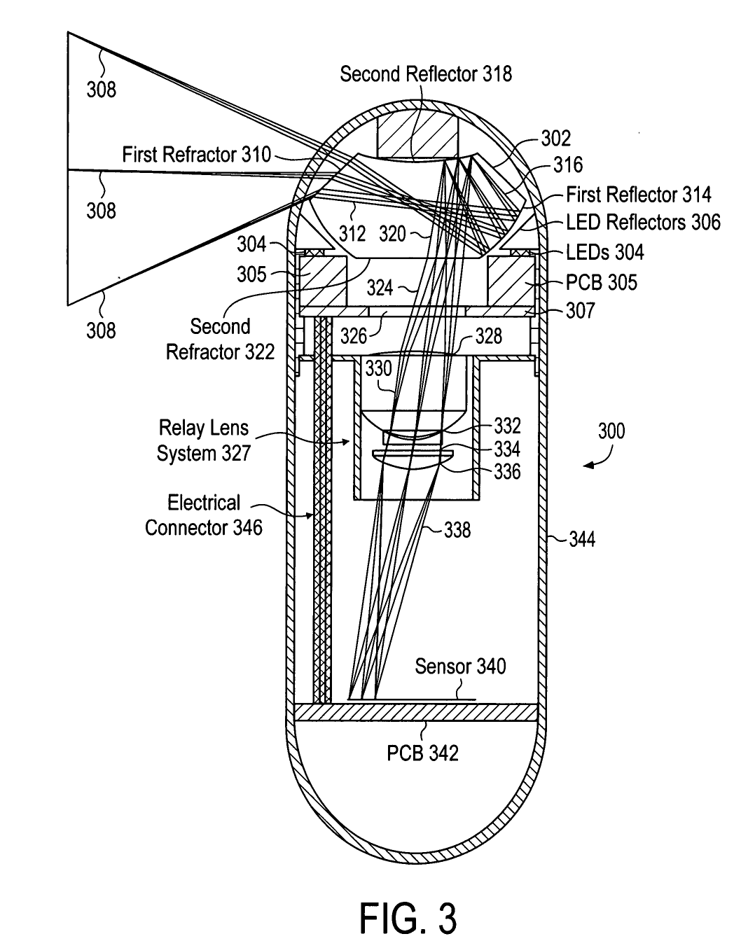

Alternatively, the capsule may contain a panoramic imaging system that comprises one or more cameras whose field of view is directed largely perpendicular to all sides of an oblong capsule so that a full 360 deg panoramic field of view is covered. A capsule camera with a panoramic annular lens (PAL) is disclosed in USPTO application ______, filed Dec. 19, 2006, entitled In Vivo Sensor with Panoramic Camera. A capsule camera 300 having a panoramic annular lens (PAL) 302, is shown schematically in FIG. 3. The lens 302 has a concentric axis of symmetry and comprises two refractive surfaces and two reflective surfaces such that incoming light passes through the first refractive surface into a transparent medium, is reflected by the first reflective surface, then by the second reflective surface, and then exits the medium through the second refractive surface.

The capsule camera 300 includes LED outputs 304 configured to illuminate outside the capsule onto a subject, such as tissue surface being imaged. The LEDs include LED reflectors 306 configured to reflect any stray LED light away from the lens 302. The purpose of the LED light rays is to reflect off of the tissue surface and into the lens 302 so that an image can be recorded. The reflectors serve to reflect any light from the light source, the LEDs, away from the lens 302 so that only light rays reflected from the tissue surface will be imaged. The LEDs are connected to printed circuit boards PCBs 305 that are connected to each other via a conductor wire or plate 307, distributing power to each LED. The lens 302 is configured to receive and capture light rays 308 that are reflected off of an outside surface, such as a tissue surface, and receives the reflected rays through a first refractor 310. The refracted rays 312 are transmitted to a first reflector 314, which transmits reflected rays 316 onto the surface of a second reflector 318. The second reflector then reflects reflected rays 320 through a second refractor 322, sending refracted rays 324 through opening 326 and into a relay lens system 327.

The system shown is a Cooke triplet relay lens, and it includes a first lens 328 for receiving the refracted rays 324 from the second refractor 322. The first lens focuses the light rays 330 onto a second lens 332. Those focused rays 334 are sent to third lens 336, which focuses rays 338 onto sensor 340. The sensor is mounted on PCB 342, which is connected to the capsule outer walls 344.

The capsule 300 further includes electrical conductor 346 connecting the PCB 342 holding the sensor to the conductor plate or wire 307. The electrical conductor 346 is configured for powering the LEDs 304 through the conductor plate 307 and PCBs 305 that hold the LEDs 304.

The PAL lens 302 produces an image with a cylindrical FOV from a point-of-view on the concentric axis. A relay image system after the PAL lens 302 forms an image on a two-dimensional light sensor 340 that may be a commonly known sensor such as a CMOS or CCD array. FIG. 3a illustrates a Cooke triplet relay lens 327. There exists other configurations that are well known in the art and include double-Gauss configurations.

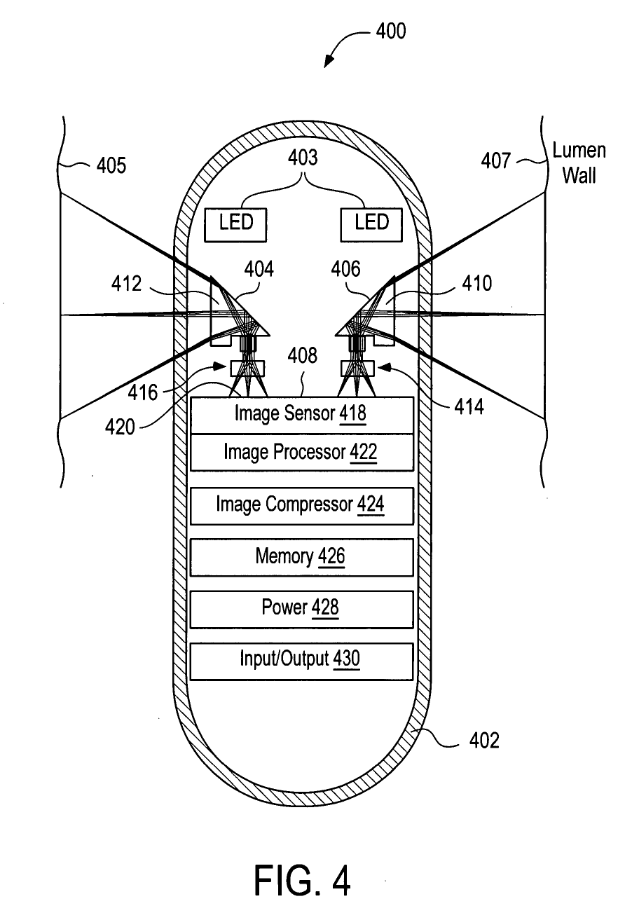

A capsule camera with a panoramic imaging system comprising multiple cameras with overlapping fields of view is disclosed in co-pending and commonly assigned U.S. application Ser. No. ______ filed on Jan. 19, 2007, entitled System and Method for In Vivo Imager with Stabilizer, and illustrated in FIG. 4. FIG. 4 illustrates 2 cameras 404, 406 that share a common image plane 408, but through the action of prisms 410 that fold the optical axes of each camera, have FOVs 409 that are substantially perpendicular to the longitudinal axis 411 of the camera. By combining a sufficient number of such cameras, such as four, the FOVs 409 may overlap so that a full 360 deg FOV about the capsule is covered. Adventitiously, the cameras may share a common image sensor 408 since the images are coplanar, and each can transfer images on their respective sensor areas 418, 420. The image sensor is configured to receive images projected on it by prisms 410, 412 and 414,416 onto image space 418,420. Image processor 422 is configured to process the images using well known processing techniques, such as storage and other processes. Image compressor 424 is configured to compress images so that less information and thus less power is required to transmit the image data. Memory 426 is for storing image data, power 428 is typically a battery for powering the components, and input/output is configured for sending image data and possibly receiving relevant data.

Because panoramic imaging systems capture images of an organ with a field of view substantially perpendicular to the tissue surface, they more readily obtain high resolution, evenly exposed, images of the organ tissues than do systems whose FOVs are centered in the forward or backward direction. Furthermore, panoramic images are more readily stitched together to form a continuous image because consecutive images captured as the capsule traverses the organ are more similar in terms of both exposure and parallax. Even without utilizing true image stitching, panoramic imaging systems facilitate image processing algorithms that reduce the number of redundant images that are stored in the capsule or transmitted wirelessly from the capsule by comparing consecutive images.

In spite of these advantages, a capsule camera with a panoramic imaging system still encounters a number of challenges in a large organ such as the colon. If the length of the capsule is less than the width of the colon, then the capsule's orientation is not well controlled and it may even tumble as it progresses through the organ. When the capsule's longitudinal axis is not parallel to the longitudinal axis of the colon, the panoramic camera's FOV will not be as nearly perpendicular to the wall of the colon, resulting in increased parallax. Furthermore, even when oriented longitudinally, the capsule will typically not be centered in the lumen so that some portions of it are closer to the camera than others. In order to maintain proper focus over a range of object distances, a number of techniques to increase the depth of field are well known. The F/# of the imaging system may be reduced. However, this reduces the diffraction-limited resolution of the system and also requires more illumination to achieve proper exposure. A mechanism for controlling the focus may be included, but the focus must be controlled independently for different viewing directions. One might utilize a plurality of cameras with different FOVs that each have an autofocus mechanism. However, such an approach will add cost, complexity, and power consumption to the system. Finally, techniques such as “wavefront coding” combine an optical filter with image post-processing to increase the depth-of-field. However, these techniques do add noise to the image during post-processing and thereby reduce the dynamic range.

An additional challenge for a capsule camera in the colon is exposure, which, for a camera without a shutter or settable aperture, becomes a problem of illumination. The side of the capsule that is farthest from the lumen wall must produce substantially more illumination than the side that is closest. While illumination about the capsule is more easily controlled than focus, spurious reflections within the capsule of a bright illumination source are more likely to produce noticeable artifacts in the image. Thus, it is desirable to limit the distance between the capsule and the lumen wall.

Finally, a variable capsule-to-tissue distance means that a frame capture rate sufficient to minimize the chance of missing tissues that are close to or touching the capsule will typically result in images of tissues that are farther from the capsule containing redundant information in consecutive images.

All of the aforementioned problems are mitigated if the capsule is maintained in the center of the colon with an orientation aligned to its direction of motion along the colon. One means of stabilizing the colon is disclosed in US patent application US2006/0178557 which describes a capsule with sacks of clay attached to either end. These sacks are covered with a smooth sacrificial layer when the capsule is swallowed, and the sacrificial layer remains intact until dissolved by the action of bacteria upon entering the colon, at which time the clay absorbs water and expands. The overall shape of the system is thus like a dumbbell and the central cylinder of the capsule is suspended in the center of the colon. The application suggests that a plurality of cameras be included in the capsule, each with a different orientation, so that a 360 deg FOV is covered.

While such a system could effectively stabilize the capsule, it has a number of shortcomings. First, a viable means of panoramic imaging is not disclosed. Given the space constraints, no more than one, or at most two, independent conventional cameras can be fit into the capsule. A system that utilizes the expansion of clay upon hydration also suffers from some potential safety issues. First, if the sacks expand prematurely in the small bowel they may place too much pressure on the organ tissues resulting in eschemia and no means of controlling the size or pressure exerted by the sacks is disclosed. Furthermore, no means of reducing the size of the sacks once they have expanded is disclosed. Thus, they may become stuck behind the ileo-cecal valve, should they deploy accidentally in the small bowel, or behind a constriction in the colon that may exist due to an abnormality, or finally they may be difficult to pass through the rectum out of the body.

Thus there exists a need in the art for a more improved system and method for stabilizing a swallowable capsule camera system for safe and effective in-vivo viewing of internal organs such as the colon that are large relative to the diameter of a capsule that is easily swallowed. As will be seen below, the invention provides such a system and a method that overcomes the problems of the prior art, and they do so in an elegant manner.

### BRIEF DESCRIPTION OF THE DRAWINGS

### Detailed Description of the Invention

Generally, the invention is directed to an in vivo camera system, where the system includes a capsule having at least one balloon configured to orient the capsule in a consistent orientation relative to an internal organ, and an imager encased within the capsule having a field of view that includes substantially all directions perpendicular to a subject tissue surface for capturing a peripheral image of tissue surface surrounding the capsule on a single image plane. The at least one balloon may also help to dilate an organ that might other wise be collapsed and folded so that the interior surface is more fully exposed and visible. The imager may include a panoramic camera encased within the capsule and configured to capture an image of tissue surface about the capsule on a single image plane. The orientation stabilizer may be configured to expand from at least two points on the capsule to stabilize the orientation of the capsule while traveling through an organ such as the colon.

The capsule may be configured to capture images while traveling through a gastrointestinal track, where the in vivo camera system operates in a first confined mode while traveling through the small intestine and in a second expanded mode while subsequently traveling through the colon, wherein the orientation stabilizer is configured to expand, when activated by the occurrence of at least one event to stabilize the orientation of the capsule while moving though the colon. An event may include the reception of a remote actuation signal, the expiration of a predetermined amount of time, or other event.

The system may include at least one reserve configured to store an expandable gas and a balloon actuator configured to release the expandable gas from the reserve and into the balloons located at opposite ends of the capsule. It also may include at least one reserve configured to store a mixture of substances that is at least partially in the liquid state, wherein the balloon actuator is configured to release at least one substance from the reserve into the balloons located at opposite ends of the capsule, wherein at least a portion of the substance released vaporizes.

In operation, prior to inflation, the system may contain a liquid or solution of liquids such that the total vapor pressure of the liquid or solution is substantially equal to a predetermined value, such that the balloon pressure upon inflation with vapor will not exceed this predetermined value.

For safety, the system may include a release valve configured to actuate when a predetermined balloon pressure is detected to deflate the balloon upon the occurrence of the predetermined pressure. It may alternatively include a release valve configured to actuate when the motion detector determines that the capsule has not progressed significantly for a predetermined period of time. It may alternatively include a release valve configured to actuate when the motion detector determines that the capsule has not progressed significantly over the course of some number of sequential image captures. It may alternatively include a release valve configured to actuate when the motion detector determines that the capsule has not progressed, or over the course of some number of sequential image captures when the capsule is impeded from movement.

The orientation stabilizer may be configured with balloons configured to inflate at opposite ends of the capsule using a chemical reaction that produces a net increase in gas molecules that is activated upon the occurrence of an event to expand the balloons and to stabilize the orientation of the capsule while moving though an organ. The chemical reaction may be triggered by the mixing of two or more chemicals. The chemical reaction may be triggered by the heating of one or more chemicals. The chemical reaction may alternatively be triggered by passing an electrical current through one or more chemicals.

In operation, a method for in-vivo imaging, may include 1) providing a device having a stabilization mechanism for stable panoramic in-vivo imaging of an internal organ onto a single image plane; 2) guiding the device within an organ using the stabilization mechanism; 3) emitting electromagnetic radiation in the wavelength range from the device; and 4) receiving reflections of the electromagnetic radiation from tissue surfaces for use in forming a panoramic image of the tissues from a field of view that includes directions perpendicular to the principle direction of travel.

Receiving reflections may include receiving reflections from a field of view that includes substantially all directions perpendicular to the direction of travel.

The system may upload to a host computer, and may first do so by performing compression on images detected by an image sensor to produce compressed image data; and then uploading the compressed image data to a host computer.

In operation, the system may perform a method for in-vivo imaging, the method including providing a device having at least one balloon for stable in-vivo imaging of an internal organ; guiding the device within an organ using the stabilization mechanism; emitting electromagnetic radiation in the wavelength range from the device; and then receiving reflections of the electromagnetic radiation from tissue surfaces for use in forming an image of the tissues. The method may inflate balloons at opposite ends of the device to stabilize the orientation of the device while moving within the organ. The process may further initiate an actuator upon the occurrence of one or more events, then inflate stabilizing balloons at opposite ends of the device by the actuator in response to initiation to stabilize the orientation of the device while moving within the organ. An event may include the passage of a predetermined period of time. An event may include the passage of a predetermined period of time that is calculated to enable inflation of the balloons when the capsule enters a subject's colon.

An event may include the reception of a remote actuator signal. An event may alternatively include be a detection by an image processor that the capsule is within the colon. Still further, an event may be composed of several sub-events, where multiple such sub-events must happen before an event is deemed to have occurred. For example, it may be desired that a balloon open upon entry to the colon. But, previous ascertainable events may be monitored and detected, such as entering the stomach, then entering the colon. Thus, waiting until after entering the stomach would serve to prevent premature expansion of the balloons prior to the stomach. Such a detection may be performed by image processing techniques that estimate the size of the organ in which the capsule resides at a particular time. For example, the capsule may measure the energy of illuminating light reflected by the surrounding organ and received by an imager relative to the energy of the illumination emitted from the capsule. Alternatively, the capsule may determine the distance of the lumen wall at regions where the fields-of-view of two cameras overlap. The greater the image overlap, as determined by image processing algorithms, the father is the object imaged. By determining the object distance at a suitable number of locations, for example four, the diameter of the lumen may be deduced.

Any of the above techniques for determining the correct moment to release the balloons may be combined. For example, image processing techniques may adequately differentiate between the small and large bowel but not between the large bowel and the stomach. However, a swallowed capsule will pass from the stomach to the small bowel to the colon. So, while this sequence of events could be detected by image processing alone, since by measuring the size of the lumen over time, the transitions from the stomach to the small bowel and from the small bowel to the large bowel can be separately identified, a further correlation with elapsed time would provide greater confidence that the capsule had in fact entered the colon and was not still in the stomach.

The process may further include releasing balloons at opposite ends of the device using a compressed gas to expand the balloons, stabilizing the orientation of the device while moving within the organ. Alternatively, the process my include releasing balloons at opposite ends of the device using a phase transition to expand the balloons, stabilizing the orientation of the device while moving within the organ.

The process my further include deflating the balloons upon certain events, such as deflating the balloons at opposite ends of the device to reduce the size of the device while moving within the organ, or alternatively deflating the balloons at opposite ends of the device in response to a change in pressure within the balloons to reduce the size of the device while moving within the organ. Time may also be a factor, where the process deflates the balloons at opposite ends of the device in response to the expiration of a predetermined period of time to reduce the size of the device while moving within the organ. Where movement can be detected, the process may deflate the balloons at opposite ends of the device in response to the detection by the capsule of a lack of movement of the capsule relative to a subject tissue surface to reduce the size of the device while moving within the organ.

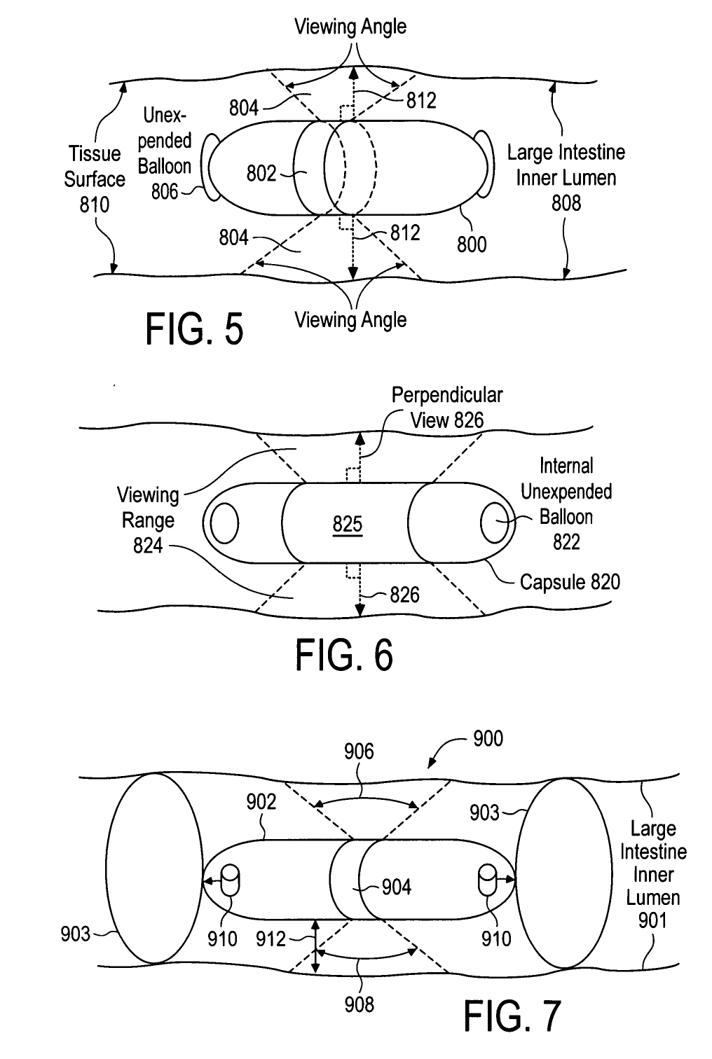

In the embodiment of FIG. 5, the imaging system, contrary to other capsule cameras, looks to the side of the capsule panoramically rather than looking in the direction of progress or backward. The window covers 360 degrees around the cylindrical portion of the capsule, so the lens is able to view the inner lumen of one section or ring of the tube-like intestine.

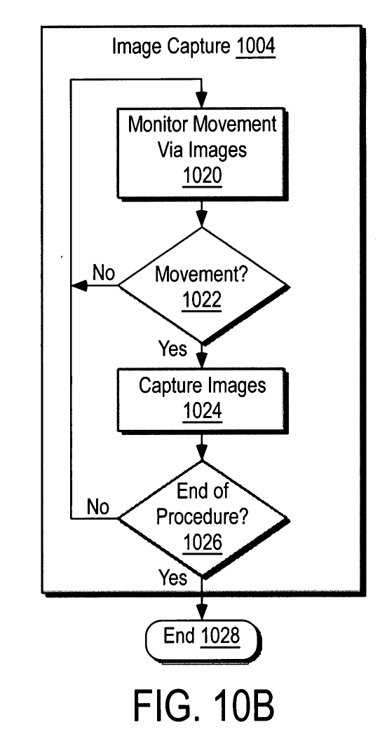

The capsule 800 includes a viewing window 802 that substantially surrounds the circumference of the capsule, giving a viewing range of substantially 360 degrees around the capsule. Also the viewing angle 804 from the window spans across the side view of the capsule. Unexpended balloons 806 are shown exterior to the capsule, but may be inside capsule, as shown in FIG. 8b and discussed below. This gives a view from the viewing window to the lumen, shown here as the large intestine inner lumen 808, onto the tissue surface 810. Given the location of the viewing window, the viewing angle 812 can include a perpendicular view 804, shown directed from the side of the capsule, as well as angles surrounding the perpendicular direction. As described further below, the images are captured through a viewing window surrounding the capsule, then onto a single image plane. The single image plane can be located on a single sensor that captures the image sent from a lens after being received by the lens. According to the invention, this unique configuration allows for images captured with an angular field of view in a range about a perpendicular direction. As discussed further below, this enables the capsule to view objects and geometries of which the tissue of interest may include that which might otherwise be obscured.

Generally, the invention is directed to an in vivo camera system, where the system includes a capsule having at least one balloon configured to orient the capsule in a consistent orientation relative to an internal organ, and a imager encased within the capsule having a field of view that includes a direction substantially perpendicular to a subject tissue surface for capturing a peripheral image of tissue surface surrounding the capsule on a single image plane. According to the embodiment of FIG. 8, the lens is able to view angles that are substantially perpendicular to the side of the capsule in directions perpendicular to the predominant direction of travel of the capsule. In prior art capsule cameras, the view is typically from the front and/or back of the capsule, in the direction or opposite to the direction of travel of the capsule through an organ, such as the esophagus or the small intestine. The prior art devices were deficient in their ability to view certain tissue features.

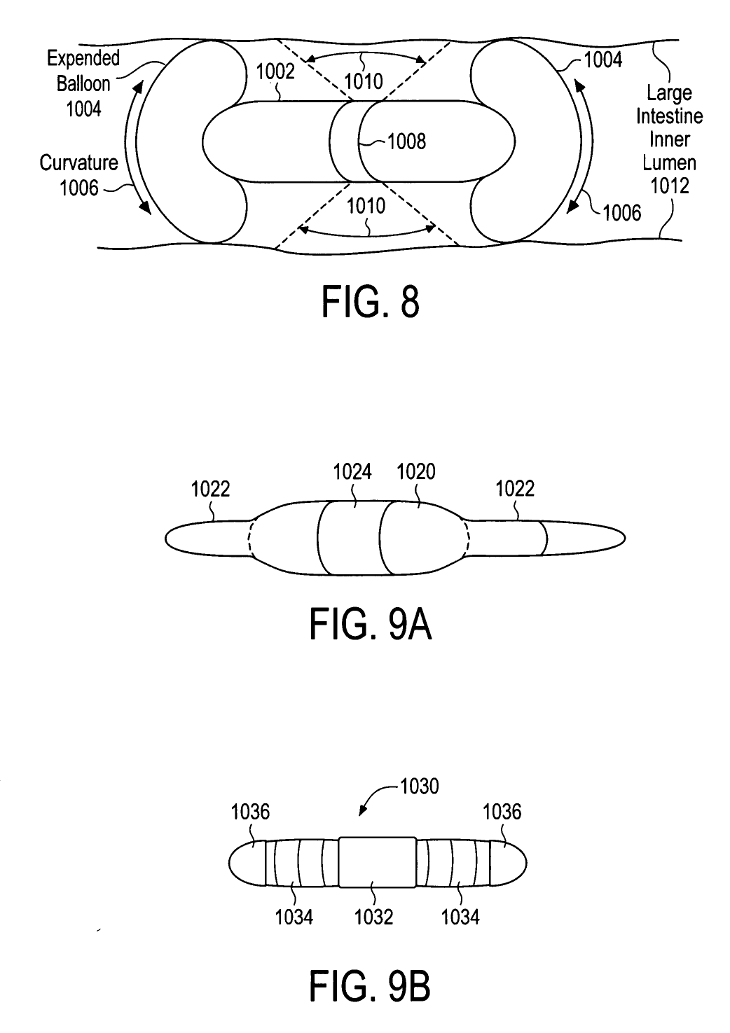

The stabilized panoramic imager helps in viewing the tissue surfaces for several reasons. The perpendicular view of the tissue surface is a direct frontal view of the tissue, in contrast to a forward or rearward view direction that results in a foreshortened perspective of the tissue surfaces. This prior art system's viewing angle can result in missing (i.e. not capturing an image of) tissue features that may be obscured behind ridges or other topological features of the tissue. When able to view from the side of the capsule according to the invention, features that may lie in sacculations are not obscured and can be imaged 136 (FIG. 2), are illustrated and discussed in further detail below. Referring to FIG. 6, an alternative embodiment to the capsule of FIG. 5 is illustrated. The capsule 820 includes internal unexpanded balloons 822, where the balloons are initially encased within the capsule before they are deployed and expanded as stabilization mechanisms. The balloons may initially be covered by a sacrificial material such as gelatin that dissolves in the GI tract. The balloons may also be covered by a protective cover that is removed just prior to balloon inflation by a mechanism. The force exerted by the balloons as they inflate might be used to remove the covers. The window 825 permits a camera behind it a viewing range 824, which includes perpendicular view 826 as well as angles on either side of the perpendicular and surrounding it.

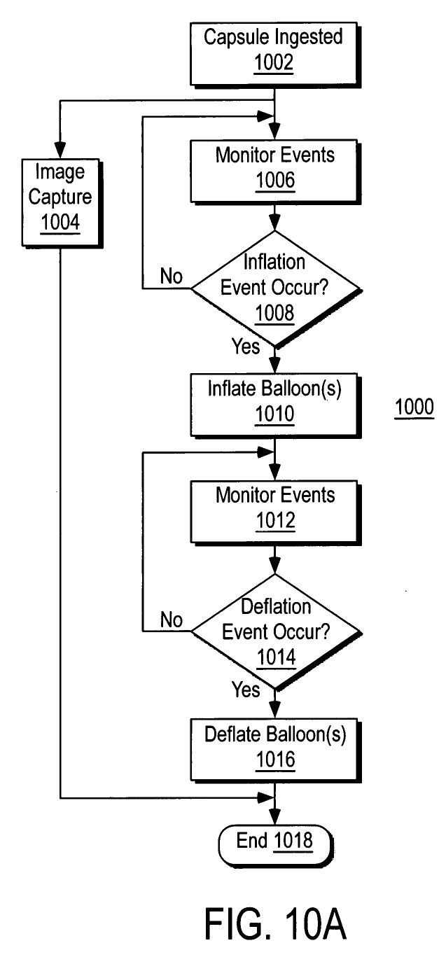

FIGS. 5 and 6 show the described camera with panoramic side view and double balloons, which are in the deflated state. FIGS. 7 and 8 show two embodiments of the described side view and double balloon structures, which are illustrated in the inflated state. In particular, FIG. 7 shows the balloon extending out from opposite ends of the capsule. FIG. 7 illustrates a system 900 within an organ, the inner lumen of a large intestine 901, that includes capsule 902 with expanded stabilizing mechanisms, the balloons 903, with the capsule having circumferential viewing window 904, affording a panoramic field of view 906. The stabilizing mechanism includes balloons 903, shown here expanded up to the inner lumen 901 of the organ, indicated as the large intestine here. When expanded, the balloons allow for a stable and consistent view of the tissue of interest, the inner lumen of the large intestine here. Here, the viewing distance 912 can be kept consistent around the entire capsule while images are being captured and parallax is reduced relative to the case of a capsule close to one side of the lumen.

Unlike other organs such as the esophagus or the small intestine, the large intestine is larger and more difficult for the capsule camera to capture images without a stabilizing mechanism. As discussed in the background, prior art devices fail for several reasons. The conventional capsules are inadequate because they are not stable in larger organs, such as the colon or large intestine. The use of sacks of clay for stabilization as described in the background raises safety concerns. The imaging systems of prior art devices are inadequate because they are not able to adequately capture a panoramic image. Including several cameras within a capsule is not practical given the space constraints. Without the ability to capture an image on a single image plane, multiple sensors and related hardware are required to capture and process the images. In contrast, the invention provides a novel and elegant device that greatly improves image capture with a panoramic imager that is able to capture images on a single image plane.

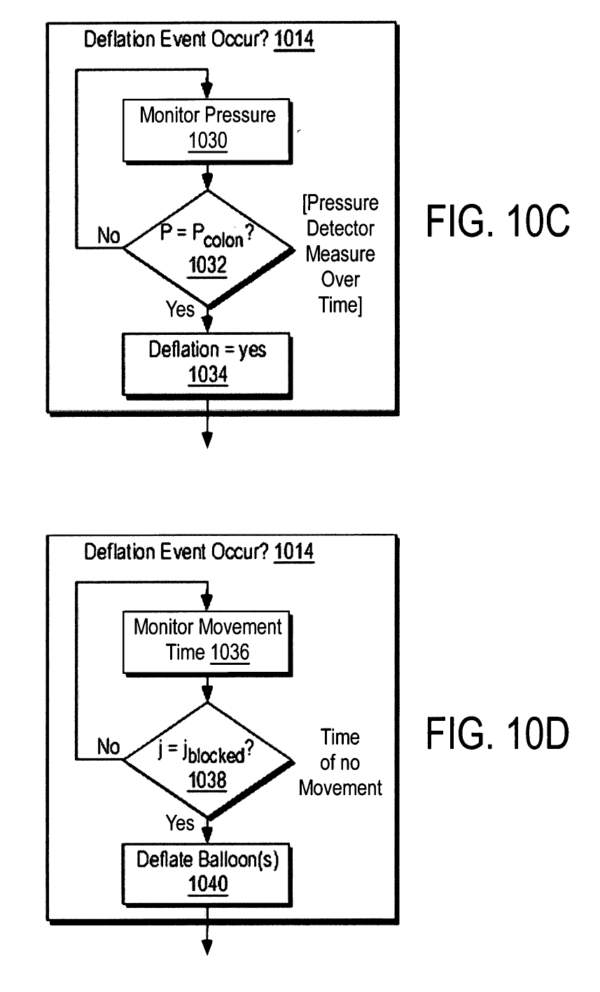

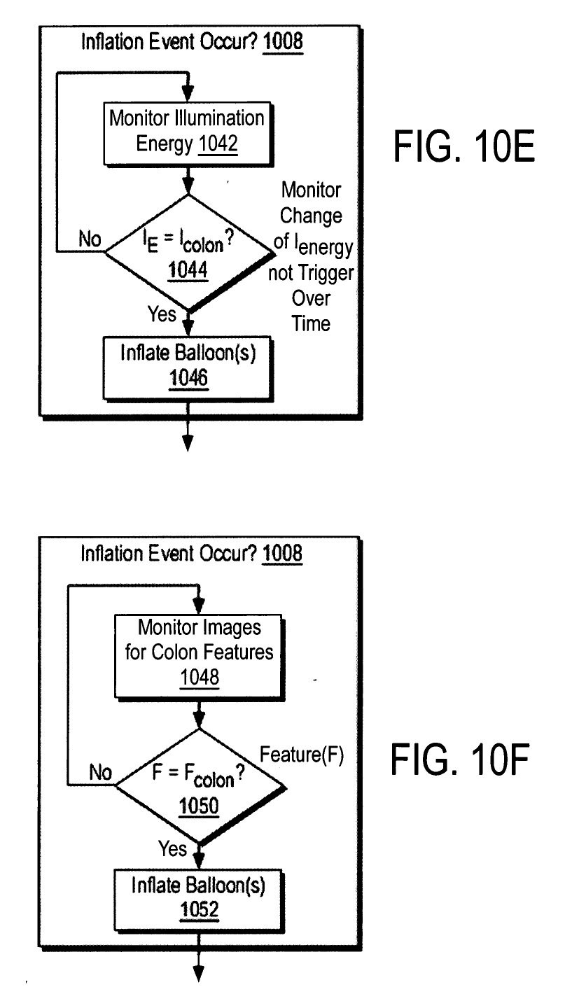

FIG. 8 shows the balloons extending partially along the sides of the capsule. The angle of the balloons in FIG. 10a may provide for an easier advance along the tract because of the normally collapsed state of the large intestine when empty (not shown in the figures). FIG. 10a shows capsule 10002, having window 1008 with viewing angle 1010 as in the above described figures, it further has expanded balloons 1004, which have a curvature 1006, that smoothens out the ends of the expanded capsule for possibly easier progression and movement along the lumen 1012. The curvature may take on different forms or shapes, but is directed to provide an improved shape to aid in easy motion through the lumen 1012. FIG. 9a illustrates an alternative embodiment of the invention, where the expanded balloon extends in the direction of the longitudinal axis of the capsule, where the balloons 1022 extend out the ends of the capsule, without expanding to increase the thickness of the capsule. This would give the window 1024 a stable configuration to avoid tumbling when traveling in a large organ, such as the large intestine. Since the diameter of the system does not increase, less of a threat is posed should the balloons inflate prematurely in the small bowel.

Those skilled in the art will understand that, given this disclosure, many different configurations are possible, perhaps with different shapes and sizes, without departing from the spirit and scope of the invention. One such example is one expanded balloon like that illustrated in FIG. 7 on one end, and another balloon expanded like that illustrated in FIG. 10b on the other end. Referring to FIG. 9b, yet another embodiment 1030 is illustrated, where intermediate portions 1034 of the capsule are expandable, with end caps 1036 extending along with the expanding balloons 1034. Again, many different embodiments are possible given this disclosure.

In one embodiment, the two balloons are coated with hydrophilic material to reduce friction with the lumen wall. Alternately, only one balloon is coated or one is coated more heavily than the other.

In one embodiment, the inflatable balloons will deflate after a certain time. This addresses the problem where the balloons are inflated too early, such as in the esophagus or small intestine, and possibly cause a blockage. The capsule will cause the balloons to automatically deflate to avoid the capsule being stuck for too long a period of time. In one embodiment, a clock circuit is able to keep track of the time even when other activities have finished, and causes deflation at that point. Alternately, upon the electronics detecting no movement for a period of time from examination of the images, the balloons will be deflated. The counter runs at a small operating current and at a low voltage and in one embodiment has a different power supply. The mechanism for deflating the capsule may be a valve configured to deflate the balloons upon a predetermined event, such as a change in pressure detected by a pressure sensing mechanism. This way, if there is some type of blockage while the capsule is traveling with the inflated balloons, the balloons can deflate to prevent continued blockage by the device. In one embodiment, the valve is normally open, so that power is required to keep the valve closed. This way, if there is a power loss, the balloons would deflate, removing a potential hazard resulting from inflated balloons that may not be able to deflate. Alternatively, the balloon may be deflated if a motion detector determines that the capsule has not progressed for some period of time, or over the course of some number of sequential image captures, as would be the case if the capsule were blocked by a constriction in the GI tract such as the ileal-cecal valve. The motion detection may be accomplished by comparing image frames captured in sequence. The greater the difference measured between two images the greater is the motion that is likely to have occurred during the interval between their times of capture. Various algorithms for motion detection are well known in the art and include the algorithms based on motion vectors or on absolute differences. Motion may be detected by a pair of pressure sensors as described below. Other forms of motion detection, for example using sonar or echo location, are possible.

There are other events that may cause the valve to open, perhaps to partially or fully deflate the balloon at times, and also events to re-inflate the balloons at a later point. For example, a timing mechanism may be incorporated to allow inflation or deflation upon predetermined time periods. A timer could be used to establish such times, and may be set upon initiation of the procedure, such as when a capsule is swallowed or inserted into a patient. After a period of time, the balloon may inflate in response to a timer setting off the inflation mechanism. The timer could also trigger the valve to deflate the balloon. The balloon may be inflated when a determination has been made that the capsule has passed from the small bowel to the cecum. If the image sensor signal intensity is nearly continually strong for some time, in relation to the illumination strength, throughout a large portion of the sensor pixels, the capsule is determined to be in a relative narrow lumen, e.g. the small intestine. If later the signal intensity, in relation to the illumination strength, from some significant fraction of the sensor pixels, drops below some threshold and remains so for some period of time, we may surmise that the capsule has entered the cecum, which, due to its greater girth, will reflect a lower fraction of the illuminating light into the camera, assuming it has a reflectivity that is not, on average, significantly larger than that of the small bowel.

The invention provides a means to use the combination of sensed light from the sensors and driving parameters from the LEDs used to illuminate tissues located about the capsule to help determine whether the capsule has moved into the large intestine. Once this is ascertained by the capsule, it can actuate the stabilizers, such as the balloons, and properly orient the capsule for optimum viewing by the camera embodied therein the capsule. The illumination energy is directly proportional to the LED drive current integrated over time. It the current is constant, then the illumination energy proportional to (driving current) X Time.

Because the large intestine is larger in size and more spacious inside than is the small intestine, more illumination is desired so that better images can be captured. This is because, after the capsule camera has entered the large intestine, the viewing distance between the lens and the tissue of interest increases. Thus, more light is needed to illuminate the tissue so that more light can be reflected back to the lens, providing more reflected light to produce an image and to get an adequate sensor reading. The image can be optimally captured as a result.

If a panoramic imaging system utilizes more than one camera with overlapping fields of view, the distance between the capsule and that portion of an object that lies in the FOV of two cameras can be determined. An image processing algorithm can determine what fraction of the total images overlap. The greater the overlap, the less the distance.

By way of example, one method for inflating and deflating the balloons according to the invention is illustrated in FIG. 10a, a general process 1000 illustrated in flow chart. In operation, a capsule is injested in step 1002. From there, two processes operate in parallel. In step 1004, image capture occurs, which can occur throughout the process while the capsule travels throughout the GI track. At the same time, a series of monitoring processes occurs beginning with step 1006, where inflation events are monitored. If an inflation event does not occur as determined in step 1008, then the process loops back and continues monitoring the events in step 1006. When an event occurs, then the process initiates the inflation process in 1010, where the balloon or balloons are inflated. After the balloons are inflated, then the process must monitor the system to watch for deflation events in step 1012. Until a deflation event occurs, the process loops back to step 1012, where deflation events continue to be monitored. Once a deflation event occurs as determined in step 1014, then the balloons are deflated in step 1016. The process ends in step 1018.

Referring back, more detailed processes within some of the individual steps of FIG. 10a are illustrated in FIGS. 10b through 10f. In FIG. 10b, a more detailed process of image capture of step 1004 is illustrated. First, the process monitors movement via images in step 1020. Then, it is determined whether there was movement in step 1022. If movement does not occur, then the process loops back to step 1020 for further monitoring. Once movement occurs, then the process proceeds to step 1024, where images are captured. This feature provides for great reduction in images captured, where images are only captured when there is movement, greatly reducing redundant images. Thus, the physician or other medical professional does not need to review as many images as otherwise required. In step 1026, it is determined whether the end of the procedure has been reached. If not, then the process returns to step 1020, where the movement of the capsule is further monitored, and the process continues. If the end of the procedure occurs, whether the capsule has completed the process and been expelled or if it is ended for any other reason, the process ends at step 1028, which corresponds to step 1018 of FIG. 10a.

Referring to FIG. 10c, a more detailed illustration of the step 1014, determining whether a deflation event has occurred, is shown. In step 1030, the pressure is monitored. This process monitors pressure as a deflation event, so that the balloon or balloons would deflate when there is an unsafe increase in pressure, indicating a blockage of some sort, or perhaps a premature inflation in a small organ such as the esophagus or a small intestine, or perhaps the capsule has entered the colon, just before it enters the large intestine, and it is stuck. If no change occurs, the process continues to monitor the pressure in step 1030. If a predetermined pressure level is detected in step 1032, such as P=P_{colon}, this indicates that the capsule has incurred a deflation event in step 1034, and the balloons will be deflated in step 1016 (FIG. 10a).

In FIG. 10d, another embodiment of a determination of whether a deflation event of step 1014 (FIG. 10a) occurs. Here, the time of movement is monitored in step 1036. Here, it is determined in step 1038 whether there has been no substantial movement of the capsule in a person's GI track. If movement occurs, then the process returns to step 1036 for further monitoring. If, however, it is determined in step 1038 that enough time has passed to be concerned, then the process deflates the balloons in step 1040, which corresponds to step 1016 of FIG. 10a. The process then ends in step 1018, FIG. 10a.

Referring to FIG. 10e, an example of a determination of whether an inflation event, step 1008 of FIG. 10a, occurs is illustrated. In step 1042, the illumination energy I_{E} required to obtain a desired image exposure is measured and monitored. In step 1043, it is determined whether the capsule is not in the stomach. If it is in the stomach, the process returns to step 1042 for monitoring. This is useful in preventing premature expansion in the stomach, preventing a false event indication. In step 1044, it is determined whether the illumination energy is at a level that indicates entry of the capsule into the colon, I_{Colon}. If not, the monitoring continues in step 1042. Once such an energy is reached, it is then determined whether the capsule is inside the small bowel in step 1045, this prevents premature inflation as well. If not in the small bowel, then the process returns to step 1042. If it is in the small bowel, then it is not likely a false read. The process then proceeds to the next step where the balloons are inflated in step 1046, corresponding to step 1010, FIG. 10a, and the process proceeds to step 1012.

Referring to FIG. 10f, another example of a determination of whether an inflation event occurs is illustrated. In step 1048, the process monitors images captured for colon features. Then, it is determined whether the capsule is in the stomach. If it is in the stomach, it returns to step 1048. If not in the stomach, the images are then compared in step 1050 to known colon images. If there are no colon images, then the process loops to step 1048 for further monitoring. then determine If an image of a colon does occur in step 1050, then it is determined whether the capsule is in the small bowel. If not in the small bowel, then the process returns to step 1048 for monitoring. If it is in the small bowel, then the process inflates the balloons in step 1052.

Referring to FIG. 10g, the process determines in a different embodiment whether an inflation event occurs. In step 1055, it is determined whether the capsule is in the small bowel. If it is not, then the process goes back until it is in the bowel. Then, the counter is set to zero in step 1056, and the overlap X between images capture by the cameras with overlapping FOVs are measured. In step 1060, it is determined whether the overlap is greater than a predetermine amount X0. If not, the process returns to step 1056. If it does, the counter is incremented in step 1062, and it is determined whether the count exceeds a predetermined count NO. If it does not, the process returns to step 1058. It does exceed NO, then the balloons are inflated in step 1066.

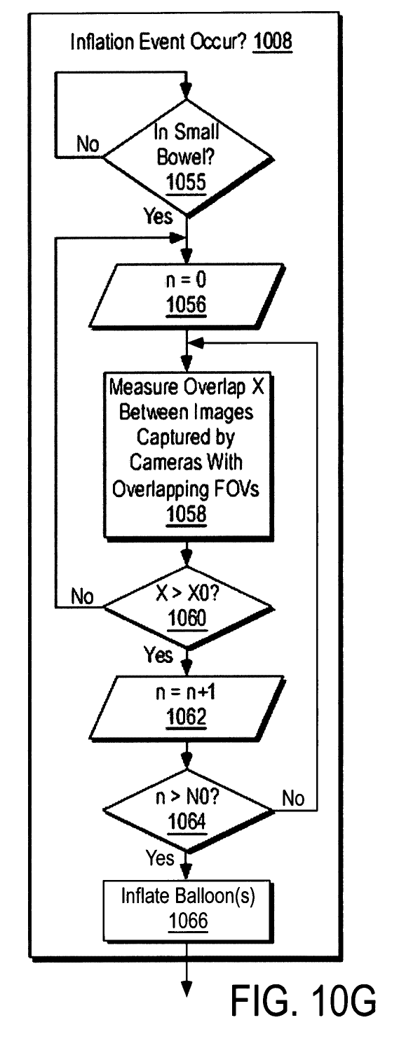

By way of example, FIG. 11 shows a cross section of a cylindrical capsule. Within the capsule are four cameras. These cameras may have separate centers of perspective C1, C2, C3, and C4 that lie in the entrance pupils of each camera. Associated with each camera is also a horizontal field of view HFOV. Each camera “faces” a different direction such that the optical axes are, in this case, separated by 90 deg. Since the HFOV of each camera exceeds 90 deg, the HFOVs overlap. Advantageously, they overlap along vertical lines within the capsule so that the horizontal extent of an object touching the capsule on the outside may be viewed in its entirety.

Also shown in FIG. 11 is a cross section of the lumen. The distances from the center of the capsule O to four points on the lumen wall I, J, K, and L in the plane of cross section are uniquely determined by the amount of overlap between adjacent images captured of the lumen. The distance OK is linearly related to the overlap x.

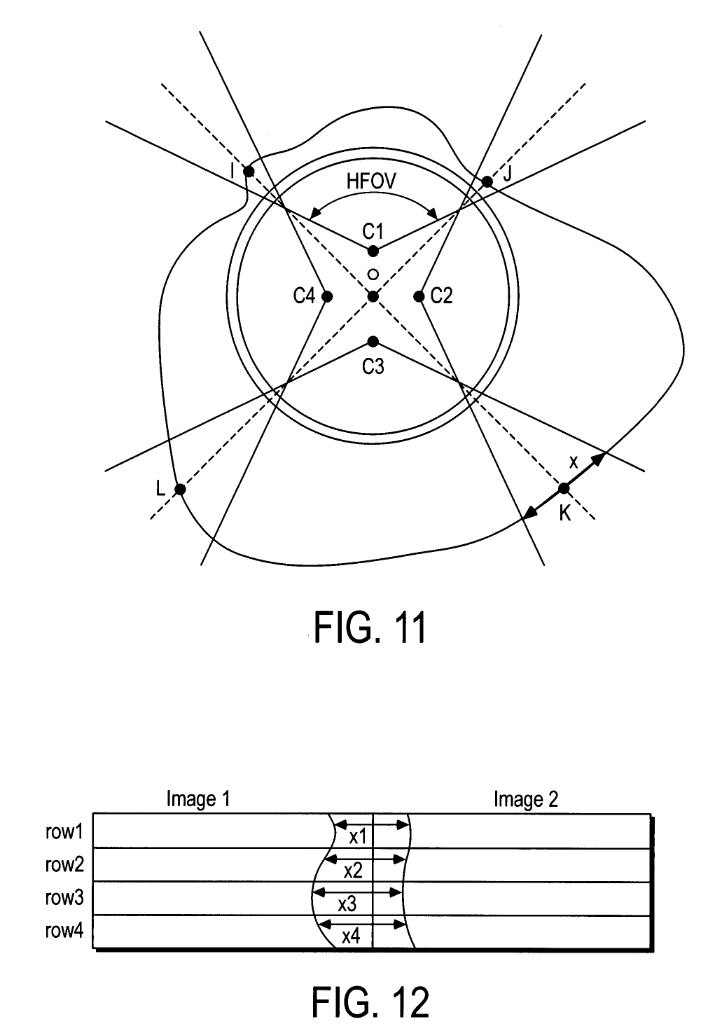

FIG. 12 shows two images captured by two adjacent cameras. The images are placed side-by-side. Only one feature of the images is shown, a line. This line might correspond to the edge of some physical feature on the lumen. Due to the non-coincident centers of perspective and the fact that the line on the lumen is not a constant distance from the capsule along its vertical extent, the line has a slightly different shape in the two images. An algorithm that determines the overlap might first divide the images into a series of horizontal bands (Four are illustrated in FIG. YY). Each band could then be translated horizontally until the best image match is found in the region where the translated bands overlap. In this simple case, that would occur when the line sections most overlap. The optimal translation distances (overlaps) for each band are labeled x1, x2, x3, and x4. Similarly, overlaps and corresponding object distances can be determined at the other three overlap regions. By considering a set of data, an estimate of the cross-sectional area of the lumen can be made. This estimate, along with previous estimates, can then be used to decide whether the capsule has entered the colon and whether to deploy the balloons.

Many different algorithms for aligning and stitching images have been developed and can be used to determine the overlap and corresponding object distances. The task is simplified by the fact that the relative physical locations and orientations of the cameras is known ahead of time. The relationship between overlap x and object distance can be calibrated in manufacturing.

Alternatively, distance judgments can be made by comparing multiple images made by a single camera at different times if the images overlap. A self consistent model of the camera orientation and position for each image along with the object shape must be deduced. This process is in general more difficult and prone to error than when the camera positions are known a priori but it is still possible in many cases.

Other mechanisms of determining that the capsule has entered the colon are possible, and those skilled in the art will understand that such variations are possible without departing from the spirit and scope of the invention, given this description.

In one embodiment, the balloons in their collapsed position cover the viewing window of the camera, protecting it from being smeared with body fluids that would obscure the view. The balloons when inflated keep the intestine walls away from the camera window, reducing the amount of fluids that will be deposited on the camera window.

The balloons are shaped so that the peristalsis force could easily act on them to move the capsule forward toward the direction of the anus. The balloons before swallowing may be covered by a digestible capsule material such as gelatin used on regular capsules to deliver drugs, so that they are not loose, in order for easy handling and swallowing.

The previous art described pressure sensor(s) are put on the surface or close to the surface of the capsule. In the case of a capsule with balloon(s) the pressure sensor could be put inside the balloon or inside the capsule but sensitive to balloon pressure. When a sensor detects a change in pressure, an image capturing sequence could be triggered. Two pressure sensors may be utilized, measuring the pressure in each of two balloons at either end of the capsule such that if a peristalsis pressure wave first triggers the one sensor and then the other, or if a pressure difference is measured between the two balloons, the capsule is likely to be moving and a picture or a series of pictures should be taken. Such pressure sensors could also be used to activate the release valve discussed above.

The above description refers to an image sensor. Other in vivo autonomous sensors may also utilize onboard memory to store all retrieved data. These sensors might be pH, pressure, or temperature sensors or other forms of chemical or bio sensors or they might perform spectroscopic measurements. These sensors may be combined in the same capsule as an image sensor or may exist in dedicated measurement capsules. As an example, the Heidelberg capsule is an existing device that is swallowed by a patient and makes measurements of GI-tract pH that are transmitted over a wireless link to an external receive antenna. The PH values measured frequency modulate the carrier while in the base station outside the body the frequency is FM decoded to get the voltage in analog form. The voltages then are translated into the PH values. Typically, the measurement is completed shortly after the capsule empties from the stomach into the duodenum. The wireless link could be eliminated if the data were stored within the capsule. The capsule would need to be retrieved after passing through the entire GI tract, however, which makes this approach less appealing than the current Heidelberg method of measuring GI tract pH. However, in general, replacing a wireless link with onboard memory enables a sensor to make measurements over a longer period of time without encumbering the patient or utilizing clinic resources during the measurement. For example, a sensor might be implanted in the body for a period of days or longer and subsequently removed, for example by surgery or through a catheter.

In another embodiment, a secondary sensor could be incorporated in the capsule, where a PH meter is used to help detect the entrance into the colon. In the stomach the acid level is usually very strong, with a PH level of between 1 and 2. In contrast, the first part of small intestine that connects directly to stomach has a PH level that drops to 5-6, which is a drop in acidity of more than one thousand times. This information could be use by the capsule system to detect of entrance of the capsule into colon. First, before the capsule goes through the stomach-small intestine interface connection, the inflation of the capsule can be disabled to avoid false detection. Next, the variation in PH values across the ileocecal valve could be observed and used to detect such an event of transition into the colon.

1. An in vivo camera system comprising:
a capsule having a stabilizing mechanism configured to orient the capsule in a consistent orientation relative to an internal organ; and
a panoramic imager encased within the capsule and configured with a field of view that includes substantially all directions perpendicular to the principle direction of in vivo camera system travel for capturing a peripheral image of tissue surface surrounding the capsule on a single image plane.

2. An in vivo camera system according to claim 1, wherein the imager is a plurality of cameras encased within the capsule and configured to capture a plurality of images of tissue surrounding the capsule on a single image plane.

3. An in vivo camera system according to claim 1, further comprising a cover that covers the stabilizing mechanism prior to deployment.

4. An in vivo camera system according to claim 3 where the cover is soluble in the gastro intestinal tract.

5. An in vivo camera system according to claim 3 where the cover is pushed off the stabilizing mechanism by a force applied by the stabilizing mechanism.

6. An in vivo camera system comprising:
a capsule having at least one balloon configured to inflate and orient the capsule in a consistent orientation relative to an internal organ wherein, upon inflation, the overall length of the in vivo camera system, in a direction substantially parallel to the predominant direction of camera motion, is increased; and
an imager encased within the capsule.

7. An in vivo camera system according to claim 6, wherein the imager is a panoramic imager encased within the capsule and configured with a field of view that includes substantially all directions perpendicular to a subject tissue surface for capturing a peripheral image of tissue surface surrounding the capsule on a single image plane.

8. An in vivo camera system according to claim 6 wherein the at least one balloon is covered by a cover prior to inflation.

9. An in vivo camera system according to claim 8 wherein the cover is soluble in the gastro intestinal tract.

10. An in vivo camera system according to claim 8 wherein the cover is pushed off at least one balloon by the force of its inflation.

11. An in vivo camera system according to claim 6 wherein at least one balloon attaches directly to the capsule body.

12. An in vivo camera system according to claim 6, wherein balloons are configured to expand at two or more separate locations on the capsule to stabilize the orientation of the capsule while traveling through the organ.

13. An in vivo camera system according to claim 6, wherein balloons are configured to expand at two ends of the capsule to stabilize the orientation of the capsule while moving though a colon.

14. An in vivo camera system according to claim 6, wherein the capsule is configured to capture images while traveling through a gastrointestinal track, where the in vivo camera system operates in a first confined mode while traveling through the small intestine and in a second expanded mode while subsequently traveling through the colon, wherein the at least one balloon is configured to expand, when activated by the occurrence of at least one event, to stabilize the orientation of the capsule while moving though the colon.

15. An in vivo camera system according to claim 6, wherein the at least one balloon inflates using a phase transition that is activated upon the occurrence of at least one event to expand the at least one balloon and to stabilize the orientation of the capsule while moving through an organ.

16. An in vivo camera system according to claim 15, wherein prior to inflation the system contains a liquid or solution of liquids such that the total vapor pressure of the liquid or solution is substantially equal to a predetermined value, such that the balloon pressure upon inflation with vapor will not exceed this predetermined value.

17. An in vivo camera system according to claim 14, wherein an event includes detection of entrance into the colon.

18. An in vivo camera system according to claim 14, wherein an event includes the expiration of a predetermined amount of time.

19. An in vivo camera system according to claim 14, wherein an event includes the reception of a remote actuation signal.

20. An in vivo camera system according to claim 6, further comprising at least one reserve configured to store an expandable gas and a balloon actuator configured to release the expandable gas from the reserve and into the at least one balloon.

21. An in vivo camera system according to claim 15, further comprising at least one reserve configured to store a mixture of substances that is at least partially in the liquid state, wherein the balloon actuator is configured to release at least one substance from the reserve into the at least one balloon, wherein at least a portion of the substance released vaporizes.

22. An in vivo camera system according to claim 6, further comprising a release valve configured to actuate when a predetermined balloon pressure is detected to deflate the at least one balloon upon the occurrence of the predetermined pressure.

23. An in vivo camera system according to claim 6, further comprising a release valve configured to actuate when the motion detector determines that the capsule has not progressed significantly for a predetermined period of time.

24. An in vivo camera system according to claim C6, further comprising a release valve configured to actuate and deflate the at least one balloon when the motion detector determines that the capsule has not progressed significantly over the course of some number of sequential image captures.

25. An in vivo camera system according to claim 6, further comprising a release valve configured to actuate and deflate the at least one balloon when the motion detector determines that the capsule has not progressed, or over the course of some number of sequential image captures when the capsule is impeded from movement.

26. An in vivo camera system according to claim 6, wherein the at least one balloon is configured to inflate using a chemical reaction to expand the at least one balloon and to stabilize the orientation of the capsule while moving though an organ.

27. An in vivo camera system according to claim 26, wherein the chemical reaction is triggered by the mixing of two or more chemicals.

28. An in vivo camera system according to claim 26 wherein the chemical reaction is triggered by the heating of one or more chemicals.

29. An in vivo camera system according to claim 26, wherein the chemical reaction is triggered by passing an electrical current through one or more chemicals.

30. A method for in-vivo imaging, comprising:
providing a device having a stabilization mechanism for stable panoramic in-vivo imaging of an internal organ onto a single image plane;
guiding the device within an organ using the stabilization mechanism;
emitting electromagnetic radiation in the wavelength range from the device; and
receiving reflections of the electromagnetic radiation from tissue surfaces for use in forming a panoramic image of the tissues from a field of view that includes substantially all directions perpendicular to the principle direction of travel.

31. Deploying the stabilization mechanism

32. A method according to claim 30, wherein receiving reflections includes receiving reflections from a field of view that includes substantially all directions perpendicular to the principle direction of travel.

33. A method according to claim 30, further comprising
uploading image data to a host computer.

34. A method according to claim 30, further comprising:
performing compression on images detected by an image sensor to produce compressed image data; and
uploading the compressed image data to a host computer.

35. A method for in-vivo imaging, comprising:
providing a device having at least one balloon for stable in-vivo imaging of an internal organ inflating the at least one balloon upon the occurrence of at least one event, wherein the overall length of the device, in a direction substantially parallel to the principle direction of camera motion, is increased;
guiding the device within an organ using the stabilization mechanism;
emitting electromagnetic radiation in the wavelength range from the device; and
receiving reflections of the electromagnetic radiation from tissue surfaces for use in forming an image of the tissues.

36. A method according to claim 35, further comprising:
inflating balloons at least two separate locations on the device to stabilize the orientation of the device while moving within the organ.

37. A method according to claim 30, further comprising:
initiating an actuator upon the occurrence of at least one event;
inflating stabilizing balloons at least two separate locations on the device by the actuator in response to initiation to stabilize the orientation of the device while moving within the organ.

38.

39. A method according to claim 37, wherein an event includes a predetermined period of time.

40. A method according to claim 37, wherein an event includes a predetermined period of time that is calculated to enable inflation of the balloons when the capsule enters a subject's colon.

41. A method according to claim 37, wherein the event is the reception of a remote actuator signal.

42. A method according to claim 37, wherein the event is the detection of a decrease in the fraction of illuminating light energy reflected from outside the capsule back into the capsule.

43. A method according to claim 42 wherein the detection is made by the image sensor.

44. A method according to claim 42 wherein the detection is made by a photodiode.

45. A method according to claim 42 wherein the illuminating light energy is derived from LED driving current.

46. A method according to claim 37, wherein the event is multiple occurrences of the predetermined conditions.

47.

48. A method according to claim 37, wherein the event is a detection by a PH meter that the capsule is within the colon.

49. A method according to claim 48, wherein the event is a detection by a PH meter the PH values fit a specific pattern that the capsule is within the colon.

50. A method according to claim 49, wherein an event includes multiple occurrences of the conditions.

51. A method according to claim 37, wherein an event includes a detection by an image processor that the capsule is within the colon.

52. A method according to claim 51 wherein the image processor determines the distance from the capsule to the lumen wall in at least one direction by determining the amount of overlap in at least a portion of two images captured by two cameras with overlapping fields of view.

53. A method according to claim 51 wherein the image processor determines the distance from the capsule to the lumen wall in at least one direction by determining the amount of overlap in at least a portion of at least two images captured by the same camera at two or more different times.

54. A method according to claim 35, further comprising:
inflating at least one balloon using a compressed gas to expand the balloons, stabilizing the orientation of the device while moving within the organ.

55. A method according to claim 30, further comprising:
inflating at least one balloon using a phase transition to expand the balloons, stabilizing the orientation of the device while moving within the organ.

56. A method according to claim 35, further comprising:
deflating the at least one balloon to reduce the size of the device.

57. A method according to claim 56, further comprising:
deflating the at least one balloon in response to a change in pressure.

58. A method according to claim 56, further comprising:
deflating the at least one balloon in response to the expiration of a predetermined period of time.

59. A method according to claim 56, further comprising:
deflating the at least one balloon in response to the detection by the capsule of a lack of movement of the capsule relative to a subject tissue.
